(19) United States
(12) Patent Application Publication
(10) Pub. No.: US 2005/0112185 A1
(43) Pub. Date: May 26, 2005

(54) Positively charged lipid and liposome with the lipid

(76) Inventors: Yong-Serk Park, Kangwon-do (KR); Hong-Sung Kim, Gyeongsangbuk-do (KR); Jae-Ho Moon, Seoul (KR); Doo-Ok Jang, Kangwon-do (KR); Yeon Heo, Seoul (KR)

Correspondence Address: KNOBBE MARTENS OLSON & BEAR LLP, 2040 MAIN STREET, FOURTEENTH FLOOR, IRVINE, CA, 92614, US

(21) Appl. No.: 10/942,629

(22) Filed: Sep. 16, 2004

Related U.S. Application Data
(63) Continuation of application No. PCT/KR20/01377, filed on Jul. 22, 2002.

(30) Foreign Application Priority Data
Mar. 20, 2002 (KR) 10-2002-0014923

(51) Int. Cl. A61K 9/127; A61K 38/04; C07K 5/04

(52) U.S. Cl. 424/450; 514/19; 560/169

(57) ABSTRACT

The present invention relates to positively charged lipids prepared by linking salt forms of acidic amino acid with a variety of lengths of hydrocarbon chain, a process for preparing the lipids, and liposomes comprising the said positively charged lipids. Positively charged lipids of the invention can be practically applied for the preparation of liposomes for gene therapy, since they provide better characteristics than the conventional positively charged lipids in terms of gene delivery efficiency and biocompatibility.

### RELATED APPLICATIONS

This application claims for the benefit of earlier filing dates under 35 U.S.C. § 365(c) of International Application No. PCT/KR02/01377 filed Jul. 22, 2002, designating the United States and claiming for the benefit of the earlier filing date under 35 U.S.C. § 365(b) of Korean Patent Application No. 2002/14923 filed Mar. 20, 2002. International Application No. PCT/KR02/01377 was published in English as WO 03/078383 A1 on Sep. 25, 2003, and is hereby incorporated herein by reference.

### BACKGROUND OF THE INVENTION

1. Field of the Invention

The present invention relates to novel positively charged lipids and liposomes comprising the positively charged lipids, more specifically, to positively charged lipids prepared by linking salt forms of acidic amino acid with a variety of lengths of hydrocarbon chain, a process for preparing the lipids, and liposomes comprising the said positively charged lipids.

2. Description of the Prior Art

It has been well known that many diseases are generally associated with genetic defects. As an approach to cure the diseases, gene therapy, which is developed for the treatment of disease by allowing the production of normal protein from a gene replacing a disease-causing gene, has been continuously explored in the art. Since the dawn of history, the gene therapy was first attempted to treat ADA(adenosine deaminase) deficiency, and its successful implementation opened an era of gene therapy, which is nowadays being extensively studied worldwide.

On the other hand, the gene delivery system for transferring a desirable foreign gene into a target cell is accompanied with biological and physicochemical means. The biological means using virus, although its gene delivery efficiency is high, has revealed several shortcomings that the introduction of viral genetic material may bring about a side-effect and the size of the delivered gene is rather limited. And, the physicochemical means uses gene gun, injection of naked DNA or liposome. Among them, the method using gene gun or injection of naked DNA is less satisfactory in a sense that the spectrum of target cells are limited, owing to the limitation of transferred region of a foreign gene to the applied spot of gene gun or injection, and the method using liposome has some defects that the gene delivery efficiency is relatively low compared to that employing virus and the expression is temporary. However, liposomes have several advantages as followings: they are capable of forming a lipid-DNA complex easily through the linkage with a gene; the gene delivery efficiency is relatively high compared to the method using gene gun or injection of gene; there is no side-effect caused by the introduction of viral genetic material; there is no limitation in the size of delivered gene; and, the expression of the gene in the target cell can be continuously increased if the liposome is used with a ligand linked with liposome. Accordingly, the application of liposome in the gene therapy is gradually increased, and widely used for the treatment of diseases such as tumor.

For the reasons as above, liposomes such as N-[1-(2,3-dioleyloxy)propyl]-N,N,N-triethylammoniumchloride(DOTMA), N-[1-(2,3-dioleyloxy)propyl]-N,N,N-trimethylammonium methylsulfate(DOTAP), 2,3-dioleyloxy-N-[2-(sperminecarboxyamide)ethyl]-N,N-dimethyl-1-propanammonium trifluoroacetate(DOSPA) and 3β3-[N-(N′N′-dimethylaminoethane)carbamoyl]cholesterol(DC-Chol) have been developed so far and have made a great contribution to gene therapy, however, the shortcoming of low efficiency of gene delivery has been regarded as a problem to be overcomed.

Under the circumstances, there are strong reasons for exploring and developing a novel liposome with a higher efficiency of gene delivery.

### SUMMARY OF THE INVENTION

One aspect of the present invention provides a positively charged lipid represented by formula (I):

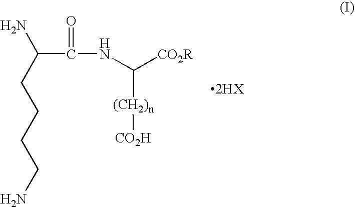

In the formula, R may be hydrogen, C₁₀-C₁₈ alkyl or C₁₀-C₁₈ alkenyl; wherein HX may be an organic or inorganic acid; and wherein n may be 1 or 2. The positively charged lipid may be lysine-aspartate-tetradecanol (KD-DM), lysine-aspartate-hexadecanol (KD-DP) or lysine-glutamate-tetradecanol (KE-DM). In the positively charged lipid, X may be F, Cl, Br, I, CF₃CO₂, HSO₄ or NH₃.

Another aspect of the present invention provides a method for preparing a positively charged lipid. The method comprises: reacting a compound of formula (V) with a compound of formula (VI) to provide a compound of formula (VII); removing R³ from the compound of formula (VII) to provide a compound of formula (VIII); reacting the compound of formula (VIII) with HX to provide a positively charged lipid of formula (I):

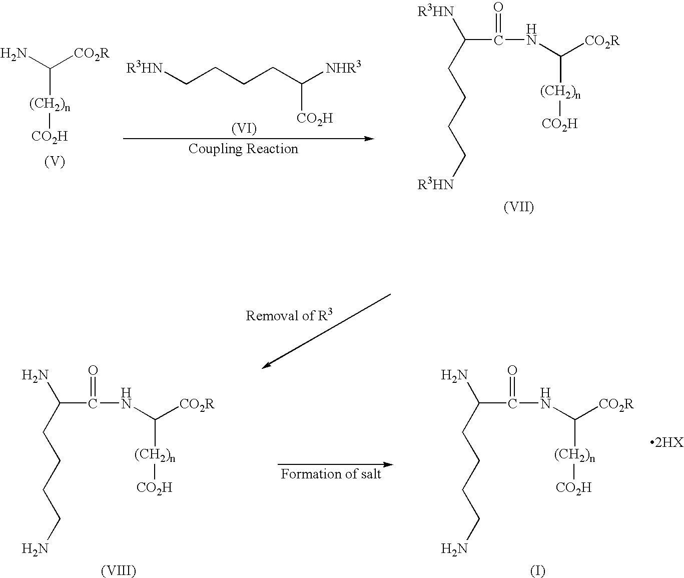

In the above reaction formulas, R may be hydrogen, C₁₀-C₁₈ alkyl or C₁₀-C₁₈ alkenyl; R³ may be an amino protecting group; HX may be an organic or inorganic acid; and wherein n may be 1 or 2. In the method, the amino protecting group may be benzyl carbamate (Cbz), p-methoxybenzyl carbamate (Moz), p-bromobenzyl carbamate, 9-fluoromethyl carbamate (FMOC), t-butyl carbamate (t-Boc) or 1-adamantyl carbamate (Adoc). In the formula (I) X may be F, Cl, Br, I, CF₃CO₂, HSO₄ or NH₃. The positively charged lipid may be lysine-aspartate-tetradecanol (KD-DM), lysine-aspartate-hexadecanol (KD-DP) or lysine-glutamate-tetradecanol (KE-DM).

In the reaction formulas, the compound of formula (V) may be provided by: esterifying an N-protected amino acid (II) with an alcohol (III) to provide a compound of formula (IV); and deprotecting the nitrogen of the compound of formula (IV).

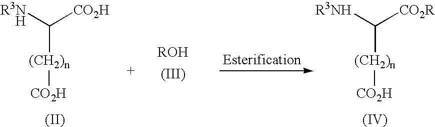

Here, R¹ may be benzyl carbamate (Cbz), p-methoxybenzyl carbamate (Moz), p-bromobenzyl carbamate, 9-fluoromethyl carbamate (FMOC), t-butyl carbamate (t-Boc) or 1-adamantyl carbamate (Adoc). In this method, esterifying of the N-protected amino acid (II) with the alcohol (III) may be carried out in the presence of an acidic catalyst selected from the group consisting of p-toluenesulfonic acid monohydrate and anhydrous toluene.

Another aspect of the present invention provides a liposome comprising a positively charged lipid of formula (I). The liposome may further comprise a cholesterol or dioleoyl phosphatidyl ethanol amine (DOPE). The positively charged lipid may be lysine-aspartate-tetradecanol (KD-DM), lysine-aspartate-hexadecanol (KD-DP) or lysine-glutamate-tetradecanol (KE-DM).

Another aspect of the present invention provides a method of preparing a liposome. The method comprises: providing a positively charged lipid of formula (I); and mixing the lipid with water or an aqueous solution. The lipid may be mixed with water or an aqueous solution along with a cholesterol or dioleoyl phosphatidyl ethanol amine (DOPE). The positively charged lipid may be lysine-aspartate-tetradecanol (KD-DM), lysine-aspartate-hexadecanol (KD-DP) or lysine-glutamate-tetradecanol (KE-DM).

Still another aspect of the present invention provides a liposome-nucleotide complex, which comprises: a liposome comprising a positively charged lipid of formula (I); and a nucleotide complex to the liposome. The liposome further comprises one or more other lipids. The liposome further comprises a cholesterol or dioleoyl phosphatidyl ethanol amine (DOPE). The nucleotide may be a DNA. The nucleotide may be a luciferase gene. The positively charged lipid may be lysine-aspartate-tetradecanol (KD-DM), lysine-aspartate-hexadecanol (KD-DP) or lysine-glutamate-tetradecanol (KE-DM).

Still another aspect of the present invention provides a method of producing a liposome-nucleotide complex. The method comprises: providing a liposome comprising a positively charged lipid of formula (I); and contacting the liposome with a nucleotide desired to complex with the liposome. The liposome further comprises a cholesterol or dioleoyl phosphatidyl ethanol amine (DOPE). The positively charged lipid may be lysine-aspartate-tetradecanol (KD-DM), lysine-aspartate-hexadecanol (KD-DP) or lysine-glutamate-tetradecanol (KE-DM).

A still further aspect of the present invention provides a method of delivering a nucleotide to a cell. The method comprises: providing a liposome-nucleotide complex comprising a liposome and a nucleotide, the liposome comprising a positively charged lipid of formula (I); and contacting the liposome-nucleotide complex with a cell to which delivery of the nucleotide may be desired. The liposome further comprises one or more other lipids. The liposome further comprises a cholesterol or dioleoyl phosphatidyl ethanol amine (DOPE). The nucleotide may be a DNA. The nucleotide may be a luciferase gene. The positively charged lipid may be lysine-aspartate-tetradecanol (KD-DM), lysine-aspartate-hexadecanol (KD-DP) or lysine-glutamate-tetradecanol (KE-DM).

### BRIEF DESCRIPTION OF THE DRAWINGS

The above and the other objects and features of the present invention will become apparent from the following descriptions given in conjunction with the accompanying drawings, in which:

FIG. 1a is a graph showing gene delivery efficiencies of KD-DM/Chol, KD-DM/Dope and KD-DM liposomes with varied amounts of DNA in 293 cell.

FIG. 1b is a graph showing gene delivery efficiencies of KD-DP/Chol, KD-DP/Dope and KD-DP liposomes with varied amounts of DNA in 293 cell.

FIG. 1c is a graph showing gene delivery efficiencies of KE-DM liposome, and KE-DM/Chol liposomes which were prepared by mixing KE-DM and cholesterol in various ratios, with varied amounts of DNA in 293 cell.

FIG. 2a is a graph showing gene delivery efficiencies of KD-DM/Chol, KD-DM/Dope and KD-DM liposomes with varied amounts of DNA in B16BL6 cell.

FIG. 2b is a graph showing gene delivery efficiencies of KD-DP/Chol, KD-DP/Dope and KD-DP liposomes with varied amounts of DNA in B16BL6 cell.

FIG. 2c is a graph showing gene delivery efficiencies of KE-DM liposome, and KE-DM/Chol liposome which is prepared by mixing KE-DM and cholesterol in a molar ratio of 7:3, with varied amounts of DNA in B16BL6 cell.

FIG. 3a is a graph showing gene delivery efficiencies of KD-DM/Chol, KD-DM/Dope and KD-DM liposomes with varied ratios of DNA to liposome in 293 cell.

FIG. 3b is a graph showing gene delivery efficiencies of KD-DP/Chol, KD-DP/Dope and KD-DP liposomes with varied ratios of DNA to liposome in 293 cell.

FIG. 3c is a graph showing gene delivery efficiencies of KE-DM and KE-DM/Chol liposomes with varied ratios of DNA to liposome in 293 cell.

FIG. 4a is a graph showing gene delivery efficiencies of KD-DM/Chol, KD-DM/Dope and KD-DM liposomes with varied ratios of DNA to liposome in B16BL6 cell.

FIG. 4b is a graph showing gene delivery efficiencies of KD-DP/Chol, KD-DP/Dope and KD-DP liposomes with varied ratios of DNA to liposome in B16BL6 cell.

FIG. 4c is a graph showing gene delivery efficiencies of KE-DM and KE-DM/Chol liposomes with varied ratios of DNA to liposome in B16BL6 cell.

### DETAILED DESCRIPTION OF THE INVENTION

The present inventors have made an effort to develop novel liposomes with a high efficiency of gene delivery, prepared positively charged lipids by employing salt forms of acidic amino acids whose amino groups are protected, and found that liposomes comprising the positively charged lipids can transfer a gene into target cell with a higher efficiency than the liposomes comprising conventional positively charged lipids.

The present invention provides novel positively charged lipids represented by the following general formula(I):

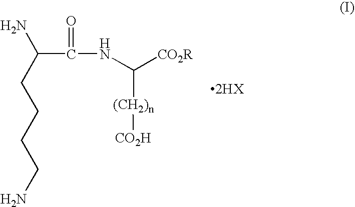

Here, R is hydrogen, C₁₀-C₁₈ alkyl or C₁₀-C₁₈ alkenyl; X is F, Cl, Br, I, CF₃CO₂, HSO₄ or NH₃; and n is 1 or 2.

A process for preparing a positively charged lipid comprises the steps of: esterifying or coupling a salt form of an acidic amino acid (II) whose amino group is protected, with an alcoholic compound (III) in the presence of an acidic catalyst of p-toluenesulfonic acid monohydrate or anhydrous toluene to obtain a compound (IV); detaching a protective group from the compound(IV) in the presence of 5 to 20% (w/w) Pd/C or hydrogen in an organic solvent of anhydrous tetrahydrofuran (THF) to obtain a compound (V); coupling the compound (V) with a compound (VI) to obtain a compound (VII); reacting the compound(VII) with Cl-dioxane solution, anhydrous dioxane or mixture thereofs at a temperature of 0 to 10° C. for 1 to 6 hrs, to remove R³ from the compound (VII) to give a compound (VIII); and, preparing a salt form of the compound(VIII) to give a positively charged lipid represented as the following general formula(I).

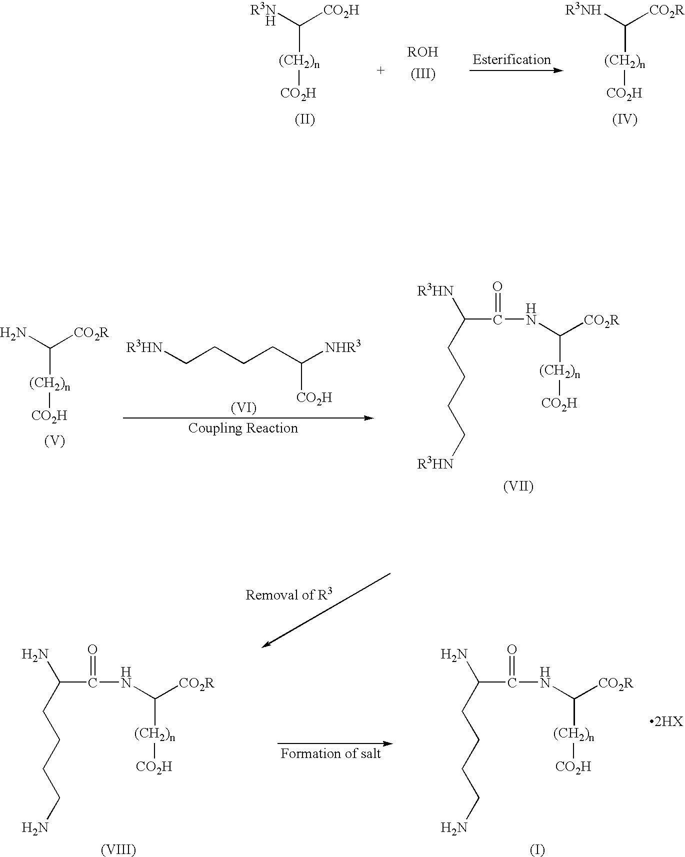

Here, R is hydrogen, C₁₀-C₁₈ alkyl or C₁₀-C₁₈ alkenyl; R¹ and R³ are benzyl carbamate(Cbz), p-methoxybenzyl carbamate(Moz), p-bromobenzyl carbamate, 9-fluoromethyl carbamate(FMOC), t-butyl carbamate(t-Boc) or 1-adamantyl carbamate(Adoc); X is F, Cl, Br, I, CF₃CO₂, HSO₄ or NH₃; and n is 1 or 2.

The process for preparing novel positively charged lipids is further illustrated in more detail, in accordance with the following steps.

Step 1: Obtainment of Compound(IV) from a Salt Form of Acidic Amino Acid

Compound(IV) is obtained by esterifying or coupling a salt form of an acidic amino acid(II) whose amino group is protected, with an alcoholic compound(III) in the presence of an acidic catalyst of p-toluenesulfonic acid monohydrate or anhydrous toluene: The acidic amino acid includes glutamic acid and aspartic acid, and the coupling reagent includes dicyclohexylcarbodiimide(DCC) and diisopropyldiimide(DIC). Furthermore, R¹ used to protect the amino group may be selected from the protective groups conventionally known in the art(see: T. W., Green et al., Protective Groups in Organic Chemistry, 2^{nd} Edition, 1991, Wiely, pp309-405, pp406-412, pp441-452), and, though not limited thereto, under an acidic reaction condition, R¹ is benzyl carbamate(Cbz), p-methoxybenzyl carbamate(Moz), p-bromobenzyl carbamate or 9-fluoromethyl carbamate(FMOC), most preferably benzyl carbamate, under a basic condition, R¹ is t-butyl carbamate(t-Boc) or 1-adamantyl carbamate(Adoc).

Step 2: Obtainment of Compound(V)

A protective group of the compound(IV) was removed in the presence of 5-20% (w/w) Pd/C or hydrogen in an organic solvent of anhydrous tetrahydrofuran(THF), to obtain a compound(V).

Step 3: Obtainment of Compound(VII) by Coupling Reaction

The compound(V) is coupled with an activated compound(VI) to obtain compound(VII): The coupling reaction is performed in an organic solvent of anhydrous dichloromethane at room temperature for 2-4 hrs, and amino group of the compound(VII) is protected with R³(=the same as R¹ defined as above).

Step 4: Obtainment of Compound(VIII)

R³ is removed by reacting the compound(VII) with Cl-dioxane solution, anhydrous dioxane or mixture thereofs at the temperature of 0-10° C. for 1-6 hrs, to obtain a compound(VIII).

Step 5: Preparation of Positively Charged Lipid

Positively charged lipid represented as general formula(I) is prepared by allowing to give a salt form of the compound(VIII), which is, as described as above, performed concurrently with the removal of R³, preferably by reacting the compound(VIII) with HCl-dioxane solution and anhydrous dioxane at 0-10° C. for 1-6 hrs, more preferably at 0° C. for 2-4 hrs, finally to prepare positively charged lipids.

Among the positively charged lipids prepared by the said process, lysine-aspartate-dodecanol, lysine-aspartate-tetradecanol, lysine-aspartate-hexadecanol and lysine-glutamate-tetradecanol, which afford high gene delivery efficiencies, are preferably employed in the invention, and lysine-aspartate-tetradecanol, lysine-aspartate-hexadecanol and lysine-glutamate-tetradecanol are most preferred positively charged lipids, which were named as “KD-DM”, “KD-DP” and “KE-DM”, respectively.

Liposome comprising positively charged lipid can be prepared by dissolving a mixture of a positively charged lipid and cholesterol or dioleoylphosphatidylethanolamine(DOPE) mixed in a molar ratio of 3:7 to 9:1 in an organic solvent, removing the organic solvent therefrom, hydrating, and mixing at a temperature of 10 to 70° C., where a mixture of chloroform and methanol(2:1, v/v) can be used as the organic solvent. In the present invention, positively charged lipids represented as general formula(I), KD-DM, KD-DP and KE-DM were preferably employed to prepare liposomes such as KD-DM/Chol, KD-DM/DOPE, KD-DP/Chol, KD-DP/DOPE, KE-DM/Chol and KE-DM/DOPE.

DNA-liposome complex was prepared by employing the liposomes thus prepared, and used to deliver a gene into target cell, and examined whether the gene can be expressed efficiently in human kidney cell. The result revealed that: liposomes comprising the positively charged lipids of the present invention provide much higher gene delivery efficiency than the conventional liposome, DOTAP/Chol which is prepared by mixing a previously known lipid of DOTAP(N-[1-(2,3-dioleoyloxy)propyl]-N,N,N-trimethylammonium methyl sulfate) with cholesterol, and provide similar effect of DNA delivery compared to DOTAP/Chol. Accordingly, positively charged lipids of the present invention can be practically applied for the preparation of liposomes for gene therapy, since they provide better characteristics than the conventional positively charged lipids in terms of gene delivery efficiency and biocompatability.

The present invention is further illustrated in the following examples, which should not be taken to limit the scope of the invention.

### Preparation of Positively Charged Lipid KD-DM using Aspartic Acid

### EXAMPLE 1-1

### Obtainment of Compound(IV)

N-Cbz-L-aspartic acid salt(210 mg, 0.8 mmol) whose amino group is protected, and tetradecanol were reacted with p-toluenesulfonic acid monohydrate(TsOH.H₂O)(50 mg, 0.26 mmol) and anhydrous toluene(10 mL) for 14 hrs at a temperature of 110° C., to obtain a compound(IV)(R═C₁₄H₂₉) with a yield of 62% by the aid of Dean-stock trap using an eluent(hexane:EtOAc=9.5:0.5, v/v).

¹H NMR(CDCl₃) d: 0.87-1.94(m, 62H), 2.85(s, 2H), 4.06(m, 4H), 4.79(m, 1H), 5.12(s, 2H), 5.7(s, 1H), 7.2-7.4(m, 5H)

### EXAMPLE 1-2

### Obtainment of Compound(V)

Protective group Cbz of compound(IV) obtained in Example 1-1 was removed by reacting the compound(300 mg, 0.46 mmol) with 10% Pd/C(58 mg, 0.55 mmol) in the presence of hydrogen in an organic solvent of anhydrous tetrahydrofuran(THF) at room temperature, to obtain a compound(V)(R═C₁₄H₂₉) with a yield of 91%.

### EXAMPLE 1-3

### Obtainment of Activated Compound(VI)

L-lysine monohydrochloride(200 mg, 1.01 mmol) and di-tert-butyl dicarbonate(477.9 mg, 2.19 mmol) were reacted in the presence of NaOH(131.5 mg, 3.29 mmol), THF(3 mL) and water(3 mL) at room temperature, to give 311 mg of compound(VI)(R³=t-BOC) with a yield of 82%. Then, the said compound, N-hydroxysuccinimide(53 mg, 0.46 mmol), DCC(103 mg, 0.5 mmol) and anhydrous dicholomethane were reacted at room temperature, finally to obtain a compound(VI) possessing active group.

### EXAMPLE 1-4

### Obtainment of Compound(VII)

Compound(V) and compound(VI) obtained in Examples 1-2 and 1-3 were mixed, reacted at room temperature for 3 hrs, to obtain a compound(VII)(R═C₁₄H₂₉) with a yield of 61% by aid of Dean-stock trap using an eluent(hexane:EtOAc=7:3, v/v).

¹H NMR(CDCl₃) d: 0.88-2.01(m, 86H), 2.79-3.05(m, 4H), 4.10(m, 4H), 4.86(m, 2H), 6.83(s, 1H), 6.9(s, 1H)

### EXAMPLE 1-5

### Preparation of Positively Charged Lipid

Compound(VII) obtained in Example 1-4 150 mg(0.16 mmol), HCl 4 mL(4.0M dioxane solution) and anhydrous dioxane 4 mL were reacted at 0° C., to prepare a positively charged lipid(“KD-DM”) with a yield of 94%.

### Preparation of Positively Charged Lipid KD-DP using Aspartic Acid

### EXAMPLE 2-1

### Obtainment of Compound(IV)

N-Cbz-L-aspartic acid salt(210 mg, 0.8 mmol) whose amino group is protected, hexadecanol(343 mg, 1.6 mmol), p-toluenesulfonic acid monohydrate(TsOH.H₂O)(50 mg, 0.26 mmol) and anhydrous toluene 10 mL were reacted for 14 hrs at 110° C., to obtain a compound(IV)(R═C₁₆H₃₃) with a yield of 65% by aid of Dean-stock trap using an eluent(hexane:EtOAc=9.5:0.5, v/v).

¹H NMR(CDCl₃) d: 0.87-1.94(m, 70H), 2.85(s, 2H), 4.06(m, 4H), 4.79(m, 1H), 5.12(s, 2H), 5.7(s, 1H), 7.2-7.4(m, 5H)

### EXAMPLE 2-2

### Obtainment of Compound(V)

Protective group Cbz of compound(IV) obtained in Example 2-1 was removed by reacting the compound(300 mg, 0.46 mmol) with 10% Pd/C(58 mg, 0.55 mmol) in the presence of hydrogen in an organic solvent of anhydrous tetrahydrofuran(THF) at room temperature, to obtain a compound(V)(R═C₁₆H₃₃) with a yield of 91%.

### EXAMPLE 2-3

### Obtainment of Compound(VII)

Compound(V) and compound(VI) obtained in Examples 2-2 and 1-3 were mixed, reacted at room temperature for 3 hrs, to obtain a compound(VII)(R═C₁₆H₃₃) with a yield of 62% by the aid of Dean-stock trap using an eluent(hexane:EtOAc=7:3, v/v).

¹H NMR(CDCl₃) d: 0.88-2.01(m, 94H), 2.79-3.05(m, 4H), 4.10(m, 4H), 4.86(m, 2H), 6.83(s, 1H), 6.9(s, 1H)

### EXAMPLE 2-4

### Preparation of Positively Charged Lipid

Compound(VII) obtained in Example 2-3 150 mg(0.16 mmol), HCl 4 mL(4.0M dioxane solution) and anhydrous dioxane 4 mL were reacted at 0° C., to prepare a positively charged lipid(“KD-DP”) with a yield of 92%.

### Preparation of Positively Charged Lipid KE-DM using Glutamic Acid

### EXAMPLE 3-1

### Obtainment of Compound(IV)

N-Cbz-L-glutamic acid salt(225 mg, 0.8 mmol) whose amino group is protected, and tetradecanol(343 mg, 1.6 mmol) were reacted with p-toluenesulfonic acid monohydrate (TsOH.H₂O)(50 mg, 0.26 mmol) and anhydrous toluene(10 mL) for 14 hrs at 110° C., to obtain a compound(IV)(R═C₁₄H₂₉) with a yield of 63% by the aid of Dean-stock trap using an eluent(hexane:EtOAc=9.5:0.5, v/v).

¹H NMR (CDCl₃) d: 0.81-1.9(m, 62H), 2.7-2.85(m, 4H), 4.04(m, 4H), 4.79(m, 1H), 5.12(s, 2H), 7.2-7.4(m, 5H)

### EXAMPLE 3-2

### Obtainment of Compound(V)

Protective group Cbz of compound(IV) obtained in Example 3-1 was removed by reacting the compound(300 mg, 0.45 mmol) with 10% Pd/C(58 mg, 0.55 mmol) in the presence of hydrogen in an organic solvent of anhydrous tetrahydrofuran(THF) at room temperature, to obtain a compound(V)(R═C₁₄H₂₉) with a yield of 93%.

### EXAMPLE 3-3

### Obtainment of Compound(VII)

Compound(V) and compound(VI) obtained in Examples 3-2 and 1-3 were mixed, reacted at room temperature for 3 hrs, to obtain a compound(VII)(R═C₁₄H₂₉) with a yield of 62% by the aid of Dean-stock trap using eluent(hexane:EtOAc=7:3, v/v).

1H NMR (CDCl3) d: 0.81-2.0(m, 86H), 2.70-3.05(m, 6H), 4.04(m, 4H), 4.8(m, 2H), 6.79(s, 1H), 6.87(s, 1H)

### EXAMPLE 3-4

### Preparation of Positively Charged Lipid

Compound(VII) obtained in Example 3-3 150 mg(0.17 mmol), HCl 4 mL(4.0M dioxane solution) and anhydrous dioxane 4 mL were reacted at 0° C., to prepare a positively charged lipid(“KD-DM”) with a yield of 92%.

### Preparation of Liposomes Comprising Positively Charged Lipids

Liposomes of DOTAP/Chol, KD-DM, KD-DM/Chol, KD-DM/DOPE, KD-DP, KD-DP/Chol, KD-DP/DOPE, KE-DM and KE-DM/Chol were prepared by employing positively charged lipids prepared in Examples 1, 2 and 3.

### EXAMPLE 4-1

### Preparation of DOTAP/Chol Liposome

DOPAT(Avanti Polar Lipids, USA) and cholesterol(Sigma Chemical Co., USA) were dissolved in a molal ratio of 1:1 in a mixed solvent of chloroform and methanol(2:1, v/v), to prepare DOTAP/Chol liposome. Then, the organic solvent was removed from the lipid containing solution by purging nitrogen gas, trace of organic solvent was subsequently removed by using a vacuum pump, hydrated with deionized distilled water, finally to prepare DOTAP/Chol liposome by the aid of a vortex mixer.

### EXAMPLE 4-2

### Preparation of KD-DM Liposome

KD-DM liposome was prepared by the same process as in Example 4-1, with an exception of dissolving KD-DM in a mixed solvent.

### EXAMPLE 4-3

### Preparation of KD-DM/Chol Liposome

KD-DM/Chol liposome was prepared by the same process as in Example 4-1, with an exception of dissolving KD-DM and cholesterol in a molal ratio of 1:1 in a mixed solvent.

### EXAMPLE 4-4

### Preparation of KD-DM/DOPE Liposome

KD-DM/DOPE liposome was prepared by the same process as in Example 4-1, with an exception of dissolving KD-DM and dioleoyl-phosphatidylethanolamine(DOPE, Doosan S. R. L., Korea) in a molal ratio of 1:1 in a mixed solvent.

### EXAMPLE 4-5

### Preparation of KD-DP Liposome

KD-DP liposome was prepared by the same process as in Example 4-1, with an exception that KD-DP was dissolved in a mixed solvent and a vortex mixer was used at 65° C.

### EXAMPLE 4-6

### Preparation of KD-DP/Chol Liposome

KD-DP/Chol liposome was prepared by the same process as in Example 4-1, with an exception that KD-DP and cholesterol were dissolved in a molal ratio of 1:1 in a mixed solvent and a vortex mixer was used at 65° C.

### EXAMPLE 4-7

### Preparation of KD-DP/DOPE Liposome

KD-DP/DOPE liposome was prepared by the same process as in Example 4-1, with an exception that KD-DP and DOPE were dissolved in a molal ratio of 1:1 in a mixed solvent and a vortex mixer was used at 65° C.

### EXAMPLE 4-8

### Preparation of KE-DM Liposome

KE-DM liposome was prepared by the same process as in Example 4-1, with an exception of dissolving KE-DM in a mixed solvent.

### EXAMPLE 4-9

### Preparation of KE-DM/Chol Liposome

KE-DM/Chol liposome was prepared by the same process as in Example 4-1, with an exception that KE-DM and cholesterol were dissolved in a molal ratio of 3:7, 4:0, 5:5, 6:4, 7:3, 8:2 or 9:1 in a mixed solvent, respectively.

### Evaluation of Gene Delivery Efficiency of Liposome

Liposomes which were prepared to comprise the varied amounts of DNA(luciferase gene) and varied mixing ratios of DNA:liposome were administered to 293 human kidney cell line(ATCC CRL-1573) and mouse melanoma cell B16BL6, and gene delivery efficiencies were evaluated and compared with one another.

### EXAMPLE 5-1

### Culture of 293 Human Kidney Cell Line

293 human kidney cells were inoculated in DMEM(Dulbecco's modified eagle medium, Gibco-BRL, USA) containing 10% (v/v) fetal bovine serum, streptomycin and penicillin, and incubated in an incubator(NAPCO, USA) under an environment of 5% (v/v) CO₂ at 37° C., which were then aliquoted in 24-well plate to reach the cell density of 1>10⁵ cell/well or 5×10⁴ cell/well, and further incubated for 24 hrs to stabilize the cells.

### EXAMPLE 5-2

### Culture of Mouse Melanoma Cell B16BL6

Mouse melanoma cells B16BL6293 were inoculated in MEM(minimum essential medium, Gibco-BRL, USA) containing 5% (v/v) fetal bovine serum, streptomycin and penicillin, and incubated in an incubator(NAPCO, USA) under an environment of 5% (v/v) CO₂ at 37° C., which were aliquoted in 24-well plate to reach the cell density of 1×10⁵ cell/well or 5×10⁴ cell/well, and further incubated for 24 hrs to stabilize the cells.

### EXAMPLE 5-3

### Evaluation of Gene Delivery Efficiency with Varied Amounts of DNA

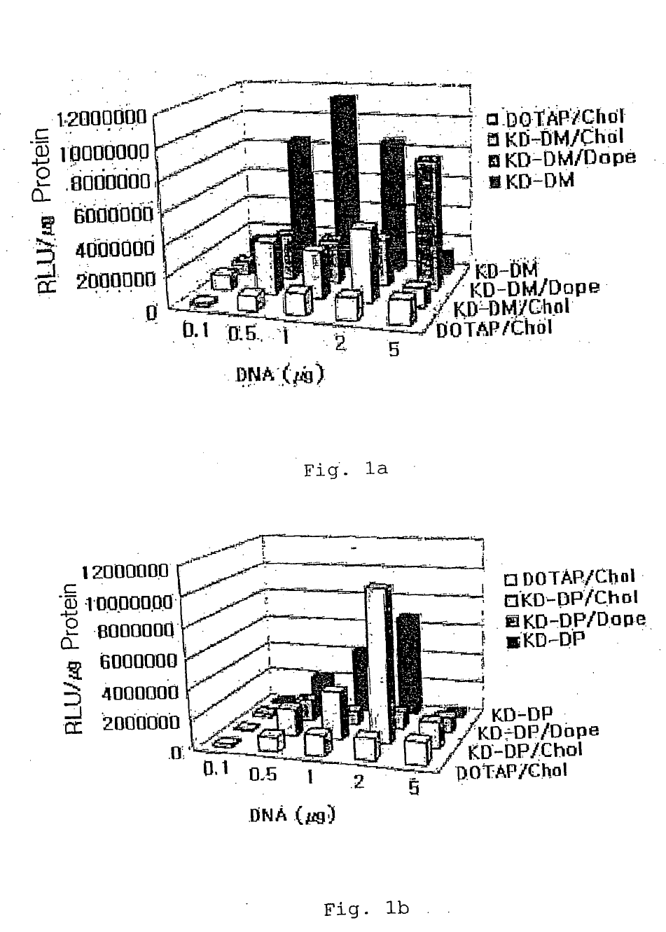

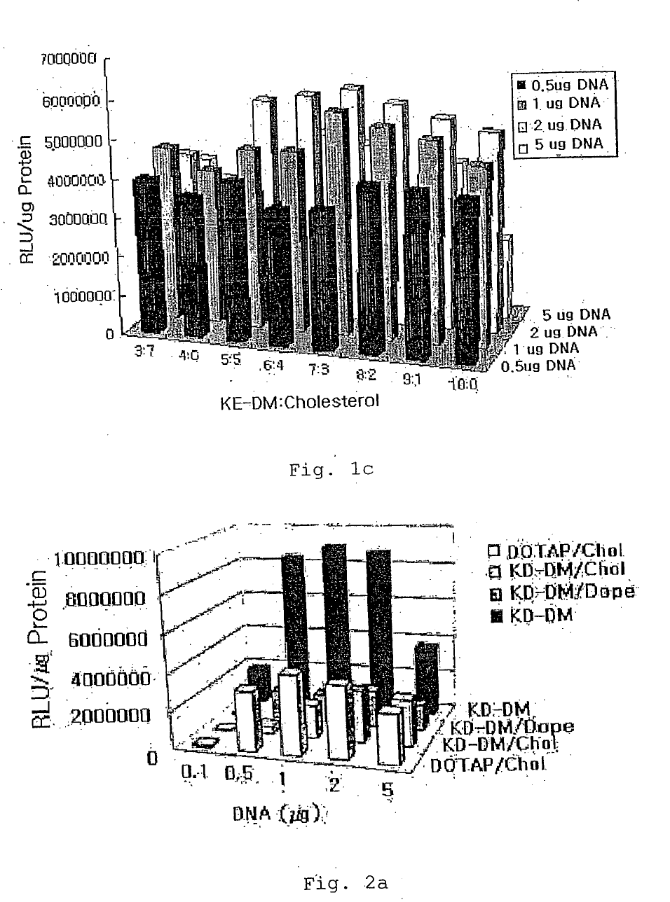

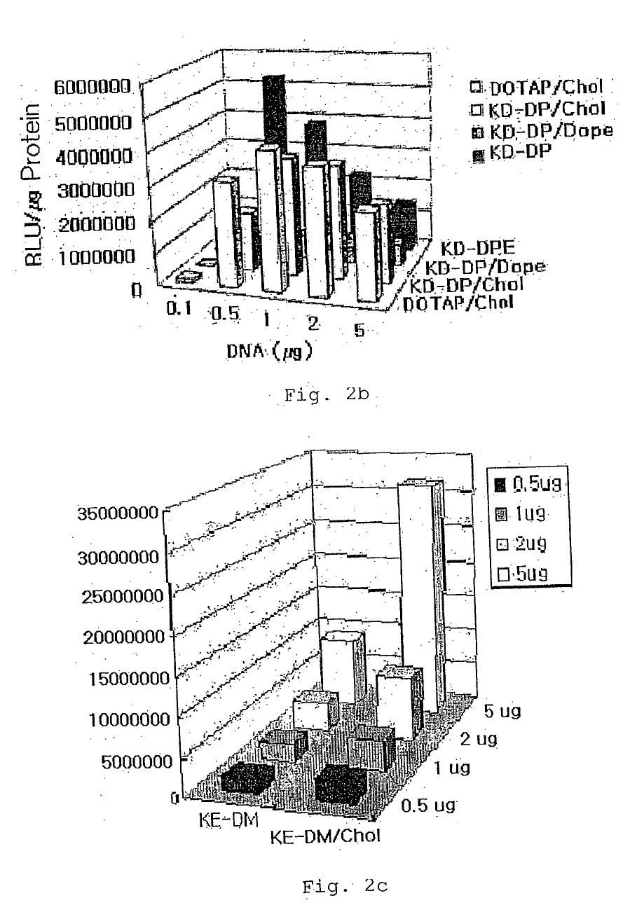

For the evaluation of gene delivery efficiency of liposomes with varied amount of DNA, DNA-liposome complex was prepared by mixing DNA and liposome(1:12, w/w) in 500 μl of serum-free medium, while varying the amounts of luciferase gene as 0.5 μg, 1 μg, 2 μg and 5 μg. 500 μl of the DNA-liposome complex was aliquoted to each well containing 293 kidney cells or B16BL6 cells, and incubated in an incubator under an environment of 5% (v/v) CO₂ at 37° C. for 4 hrs, then changed the medium to a fresh medium containing serum and incubated for 48 hrs. After incubation, media were discarded, cells were lysed by adding 100 μl of Luciferase cell culture lysis reagent(Promega, USA), and centrifuged at 14000 rpm for 10 sec. And then, 10 μl of supernatant was mixed with 20 μl of a substrate of luciferase, and luminescence was measured by using Luminometer(Mini lumat LB9506, EG&G BERTHOLD). Proteins in each well were quantitated by DC protein assay(Bio-rad, USA)(see: FIGS. 1a-1c and 2a-2c).

FIGS. 1a and 1b are graphs showing gene delivery efficiencies of KD-DM/Dope and KD-DM liposomes, and KD-DP/Dope and KD-DP liposomes with varied amounts of DNA in 293 cells, respectively, and FIG. 1c is a graph showing gene delivery efficiencies of KE-DM liposome, and KE-DM/Chol liposomes prepared by mixing KE-DM and cholesterol in various ratios, with varied amounts of DNA in 293 cells.

As shown in FIGS. 1a and 1b, luciferase gene delivery efficiencies of liposomes comprising positively charged lipids were higher than that of conventional DOTAP/Chol liposome. Comparison with the conventional DOTAP/Chol revealed that the amounts of DNA showing the highest gene delivery efficiency of each liposome were: 1 μg for KD-DM, 2 μg for KD-DM/Chol, 5 μg for KD-DM/DOPE, 2 μg for KD-DP, 2 μg for KD-DP/Chol, 2 μg for KE-DM, and 2 μg for KE-DM/Chol. Especially, as shown in FIG. 1c, the mixing ratio of KE-DM to cholesterol showing the highest gene delivery efficiency of each liposome was 7:3(in molar ratio).

FIGS. 2a and 2b are graphs showing gene delivery efficiencies of KD-DM/Chol, KD-DM/Dope and KD-DM liposomes, and KD-DP/Chol, KD-DP/Dope and KD-DP liposomes with varied amounts of DNA in B16BL6 cells, respectively, and FIG. 2c is a graph showing gene delivery efficiencies of KE-DM liposome and KE-DM/Chol liposome which is prepared by mixing KE-DM and cholesterol in a molar ratio of 7:3, with varied amounts of DNA in B16BL6 cell.

As shown in FIGS. 2a and 2b, among the positively charged lipids of the present invention, positively charged liposomes using aspartic acid showed gene delivery efficiencies lower than the conventional DOTAP/Chol liposome in case of cholesterol-bound liposome, and higher than the conventional DOTAP/Chol in case of cholesterol-unbound liposome. To the contrary, as shown in FIG. 2c, in case of positively charged liposomes using glutamic acid, cholesterol-bound liposome showed gene delivery efficiencies higher than cholesterol-unbound liposome. And, the amounts of DNA showing the highest gene delivery efficiency of each liposome were: 1 μg for KD-DM, 0.5 μg for KD-DP and 5 μg for KE-DM/Chol.

### EXAMPLE 5-4

### Measurement of Gene Delivery Efficiency with Varied Mixing Ratio of DNA:Liposome

For the evaluation of gene delivery efficiency with varied mixing ratio of DNA:liposome, DNA-liposome complexes were prepared by the same method as in Example 5-3 except that 1 μg of luciferase gene was used and the mixing ratios of DNA:liposome were 1:3, 1:6, 1:9, 1:12 and 1:18(w/w), and then, added to 293 kidney cells and incubated for 48 hrs. After incubation, the media were discarded, the cells were lysed by adding 100 μl of Luciferase cell culture lysis reagent(Promega, USA), then centrifuged at 14000 rpm for 10 sec. Subsequently, 10 μl of supernatant was mixed with 20 μl of a substrate of luciferase, and luminescence was measured by using Luminometer(Mini lumat LB9506, EG&G BERTHOLD) and proteins in each well were quantitated by DC protein assay(Bio-rad, USA).

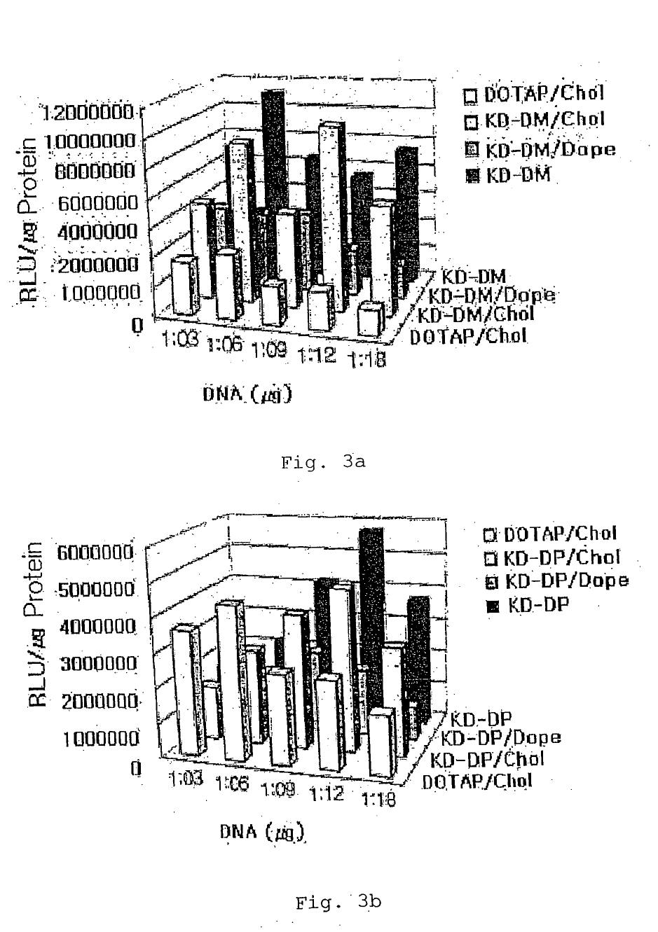

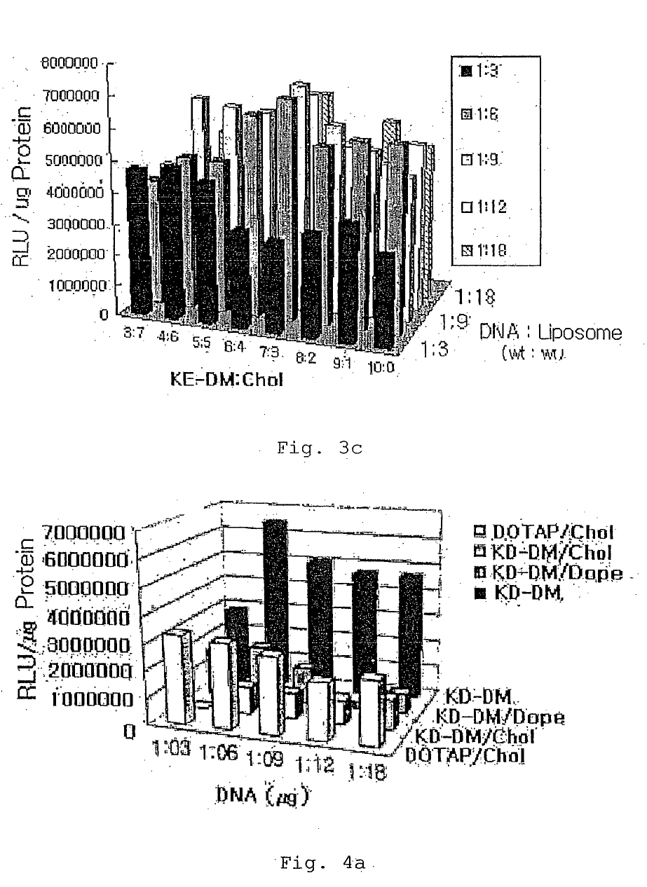

FIGS. 3a, 3b and 3c are graphs showing gene delivery efficiencies of KD-DM/Chol, KD-DM/Dope and KD-DM liposomes, KD-DP/Chol, KD-DP/DOPE and KD-DP liposomes, and KE-DM and KE-DM/Chol liposomes with varied ratios of DNA to liposome in 293 cells, respectively.

As shown in FIGS. 3a and 3b, the ratios of DNA to liposome prepared by employing aspartic acid showing the highest gene delivery efficiency were: 1:6(w/w) for KD-DM, 1:12(w/w) for KD-DM/Chol, 1:6(w/w) for KD-DM/DOPE, 1:12(w/w) for KD-DP, and 1:12(w/w) for KD-DP/Chol. As shown in FIG. 3c, in the case of liposome prepared by employing glutamic acid, the mixing ratio showing the highest efficiency was 7:3(in molar ratio) of KE-DM:cholesterol and 1:9(w/w) of DNA:liposome.

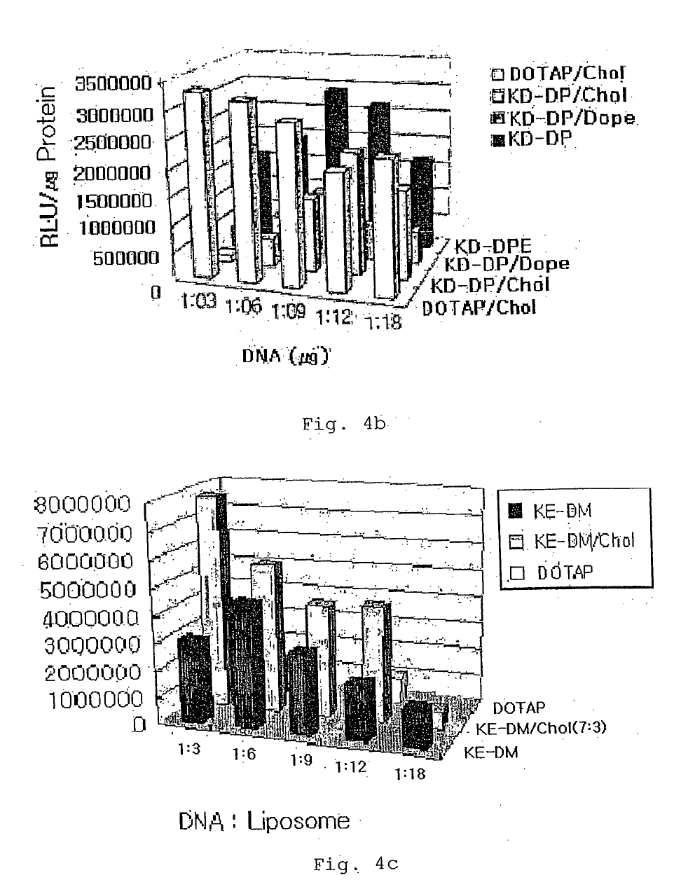

FIGS. 4a, 4b and 4c are graphs showing gene delivery efficiencies of KD-DM/Chol, KD-DM1Dope and KD-DM liposomes, KD-DP/Chol, KD-DP/DOPE and KD-DP liposomes, and KE-DM and KE-DM/Chol liposomes with varied ratios of DNA to liposome in B16BL6 cells, respectively.

As shown in FIGS. 4a and 4b, the ratios of DNA to liposome prepared by employing aspartic acid showing the highest gene delivery efficiency were: 1:6(w/w) for KD-DM and 1:9(w/w) for KD-DP. As shown in FIG. 4c, in the case of liposome prepared by employing glutamic acid, the ratio showing the highest efficiency was 7:3(in molar ratio) of KE-DM:cholesterol and 1:3(w/w) of DNA:liposome.

As clearly illustrated and demonstrated as above, the present invention provides positively charged lipids prepared by linking salt forms of acidic amino acid with a variety of lengths of hydrocarbon chain, a process for preparing the lipids, and liposomes comprising the said positively charged lipids. Positively charged lipids of the invention can be practically applied for the preparation of liposomes for gene therapy, since they provide better characteristics than the conventional positively charged lipids in terms of gene delivery efficiency and biocompatibility.

While the present invention has been shown and described with reference to the particular embodiments, it will be apparent to those skilled in the art that many changes and modifications may be made without departing from the spirit and scope of the invention as defined in the claims. Accordingly, the substantial scope of the present invention is defined as the attached claims and their equivalents.

1. A positively charged lipid represented by formula (I): 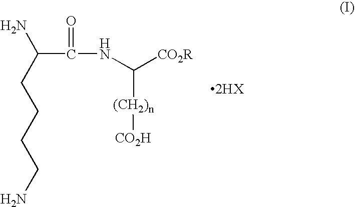
wherein R is hydrogen, C₁₀-C₁₈ alkyl or C₁₀-C₁₈ alkenyl;
wherein HX is an organic or inorganic acid; and
wherein n is 1 or 2.

2. The positively charged lipid of claim 1, wherein the positively charged lipid is lysine-aspartate-tetradecanol (KD-DM), lysine-aspartate-hexadecanol (KD-DP) or lysine-glutamate-tetradecanol (KE-DM).

3. The positively charged lipid of claim 1, wherein X is F, Cl, Br, I, CF₃CO₂, HSO₄ or NH₃.

4. A method for preparing a positively charged lipid, the method comprising:
reacting a compound of formula (V) with a compound of formula (VI) to provide a compound of formula (VII);
removing R³ from the compound of formula (VII) to provide a compound of formula (VIII);
reacting the compound of formula (VIII) with HX to provide a positively charged lipid of formula (I): 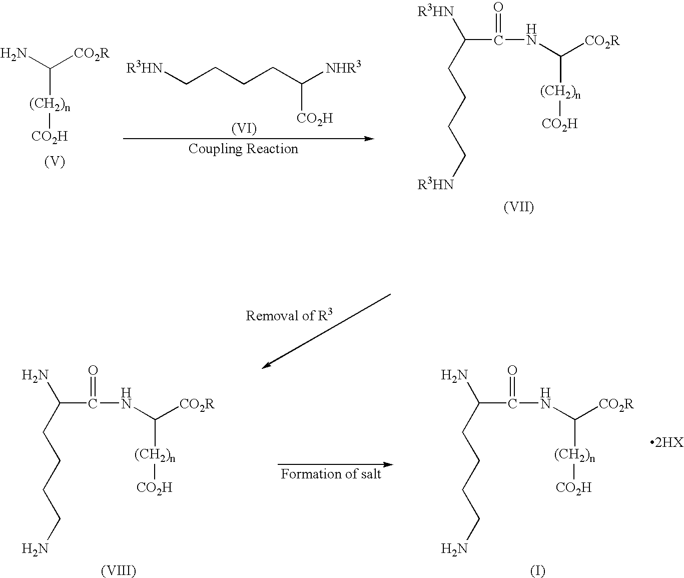
wherein R is hydrogen, C₁₀-C₁₈ alkyl or C₁₀-C₁₈ alkenyl;
wherein R is an amino protecting group;
wherein HX is an organic or inorganic acid; and
wherein n is 1 or 2.

5. The method of claim 4, wherein the amino protecting group is benzyl carbamate (Cbz), p-methoxybenzyl carbamate (Moz), p-bromobenzyl carbamate, 9-fluoromethyl carbamate (FMOC), t-butyl carbamate (t-Boc) or 1-adamantyl carbamate (Adoc).

6. The method of claim 4, wherein X is F, Cl, Br, I, CF₃CO₂, HSO₄ or NH₃

7. The method of claim 4, wherein the positively charged lipid is lysine-aspartate-tetradecanol (KD-DM), lysine-aspartate-hexadecanol (KD-DP) or lysine-glutamate-tetradecanol (KE-DM).

8. The method of claim 4, wherein the compound of formula (V) is provided by:
esterifying an N-protected amino acid (II) with an alcohol (III) to provide a compound of formula (IV); and 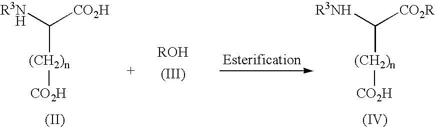
deprotecting the nitrogen of the compound of formula (IV);
wherein R¹ is benzyl carbamate (Cbz), p-methoxybenzyl carbamate (Moz), p-bromobenzyl carbamate, 9-fluoromethyl carbamate (FMOC), t-butyl carbamate (t-Boc) or 1-adamantyl carbamate (Adoc).

9. The method of claim 8, wherein esterifying of the N-protected amino acid (II) with the alcohol (III) is carried out in the presence of an acidic catalyst selected from the group consisting of p-toluenesulfonic acid monohydrate and anhydrous toluene.

10. A liposome comprising a positively charged lipid of formula (I): 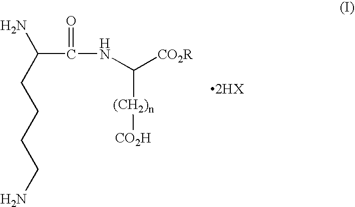
wherein R is hydrogen, C₁₀-C₁₈ alkyl or C₁₀-C₁₈ alkenyl;
wherein HX is an organic or inorganic acid; and
wherein n is 1 or 2.

11. The liposome of claim 10, further comprising a cholesterol or dioleoyl phosphatidyl ethanol amine (DOPE).

12. The liposome of claim 10, wherein the positively charged lipid is lysine-aspartate-tetradecanol (KD-DM), lysine-aspartate-hexadecanol (KD-DP) or lysine-glutamate-tetradecanol (KE-DM).

13. The liposome of claim 10, wherein X is F, Cl, Br, I, CF₃CO₂, HSO₄ or NH₃.

14. A method of preparing a liposome, comprising:
providing a solution of a positively charged lipid of formula (I) in an organic solvent: 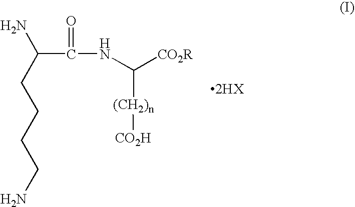
wherein R is hydrogen, C₁₀-C18 alkyl or C₁₀-C₁₈ alkenyl,
wherein HX is an organic or inorganic acid, and
wherein n is 1 or 2;
removing the organic solvent from the solution; and
thereafter mixing the lipid with water or an aqueous solution.

15. The method of claim 14, wherein the solution further comprises a cholesterol or dioleoyl phosphatidyl ethanol amine (DOPE), and wherein the lipid is mixed with water or an aqueous solution along with the cholesterol or dioleoyl phosphatidyl ethanol amine (DOPE).

16. The method of claim 14, wherein the positively charged lipid is lysine-aspartate-tetradecanol (KD-DM), lysine-aspartate-hexadecanol (KD-DP) or lysine-glutamate-tetradecanol (KE-DM).

17. The method of claim 14, wherein X is F, Cl, Br, I, CF₃CO₂, HSO₄ or NH₃.
